(19) United States
(12) Patent Application Publication
(10) Pub. No.: US 2018/0133345 A1
(43) Pub. Date: May 17, 2018

(54) Nano-Devices for Detection and Treatment of Cancer

(71) Applicant: Nanoco Technologies Ltd., Manchaster (GB)

(72) Inventor: Imad Naasani, Manchester (GB)

(21) Appl. No.: 15/809,664

(22) Filed: Nov. 10, 2017

Related U.S. Application Data
(60) Provisional application No. 62/422,714, filed on Nov. 16, 2016.
(60) Provisional application No. 62/422,480, filed on Nov. 15, 2016.

(51) Int. Cl. A61K 49/00; C07K 16/32; G01N 33/574; G01N 33/58; C07K 16/28; C09K 11/62

(52) U.S. Cl. CPC A61K 49/0067; A61K 49/0019; A61K 49/0058; A61K 49/0056; C07K 16/32; G01N 33/5748; G01N 2500/04; C07K 16/2863; C07K 16/2827; C09K 11/62; A61K 49/0052; A61K 49/0054; C07K 2317/24; G01N 33/588

(57) ABSTRACT

Compositions and methods are described for detecting and treating conditions including cancer with target specific quantum dot nano-devices. In some aspects, nanoparticle conjugates are provided having multiple target specificities and include surface modified, water soluble quantum dot (QD) nanoparticles each of which are chemically conjugated to at least two different target specific ligands.

### REFERENCE TO RELATED APPLICATIONS

This application claims priority based on U.S. Provisional Application Ser. No. 62/422,480 filed Nov. 15, 2016, and U.S. Provisional Application Ser. No. 62/422,714 filed Nov. 16, 2016, both of which are incorporated herein by reference their entireties.

### FIELD OF THE INVENTION

This disclosure relates generally to compositions and methods for the detection and treatment of cancer using conjugated quantum dot nanoparticles.

### BACKGROUND OF THE INVENTION

Without limiting the scope of the invention, its background is described in connection with existing targeting moieties for detection and treatment of tumorous tissue. Recent figures released by the Centre for Disease Control and Prevention (CDC) show that cancer has surpassed Cardio Vascular Disease (CVD) as the leading cause of death in many countries around the world. It is known that once a tumor passes the latency period of growth, it grows and invades exponentially. Early diagnosis and treatment of cancers are major determinants for survival rates and remission, particularly in those cancers with a poor prognosis such as pancreatic and brain cancers. Of these, pancreatic cancer is highly lethal and effective diagnostic and therapeutic strategies are still urgently needed to improve survival rates. Adenocarcinoma of the exocrine pancreas is one of the most devastating cancers, largely due to late presentation of recognizable symptoms. While only the 9^{th} or 10^{th} most commonly diagnosed cancer, pancreatic cancer is the fourth leading cause of cancer deaths in the United States. As of 2016, the 5 year survival rate for pancreatic cancer is only 7%. The median survival for untreated pancreatic cancer is 3 months from diagnosis. Surgery remains the primary treatment, but only a small percentage of patients present with localized disease that makes surgery a suitable option. Staging and clean surgery of pancreatic tumors is challenging due to the soft and unresectable nature of the tumors.

Current imaging approaches for pancreatic cancer are based on pre-screening for high risk groups having disseminated disease using computed tomography (CT) scans. The high risk group is then further examined for pancreatic cysts using ultrasound endoscopy (EUS, Endoluminal Ultrasonography Endoscopy) or X-ray (Endoscopic Retrograde Cholangiopancreatography (ERCP)). These methods both suffer from lack of specificity and high rate of false interpretation. Imaging devices like magnetic resonance imaging (MRI), X-ray, CT and ultrasound are indirect and lack precision demarcation capabilities that would permit adequate resection. Endoscopic devices cannot detect specific cell types without labelling.

There is a new interest in using fluorescence imaging to help surgeons identify and remove small residual tumour deposits difficult to visualise. See e.g. Nguyen Q T, Tsien R Y. Fluorescence-guided surgery with live molecular navigation—a new cutting edge. Nat Rev Cancer. 13(9) (2013) 653-62; Saccomano M et al. Preclinical evaluation of near-infrared (NIR) fluorescently labeled cetuximab as a potential tool for fluorescence-guided surgery, Int. J Cancer 139 (10) (2016) 2277-89. In addition, staging and surgical removal of pancreatic tumours are challenging. Problematically, fluorescent dyes are limited by rapid clearance, fast fading, fast metabolic degradation and low signal intensity.

From the foregoing, it appeared to the present inventor that improved tumor specific compositions were required.

### BRIEF SUMMARY OF THE INVENTION

In certain embodiments, a nanoparticle conjugates (nano-devices) are provided having multiple target specificities. In some aspects the conjugates include surface modified, water soluble quantum dot (QD) nanoparticles each of which are chemically conjugated to at least two different target specific ligands. In certain embodiments, the target specific ligands include ligands specific for binding to target moieties including proteins and carbohydrates overexpressed on tumors.

In certain embodiment the targets include one or more of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand. In particular embodiments, the surface modified, water soluble QD nanoparticle is chemically conjugated to at least two ligands having specificities for targets including EGRF and PD-L1. An example of a ligand having target specificity for EGRF is the monoclonal antibody cetuximab. An example of a ligand having target specificity for PD-L1 is the monoclonal antibody atezolizumab.

In other embodiments a mixture of nanoparticle conjugates (nano-devices) is provided including at least two populations of nano-devices: a first population comprising first therapeutic antibodies directed to a first target attached to a surface modified, water soluble QD nanoparticle and a second population comprising second therapeutic antibodies directed to a second target attached to a surface modified, water soluble QD nanoparticle. The target specific antibodies may be specific for target proteins and carbohydrates overexpressed on tumors including EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

In certain embodiments, the surface modified non-toxic, water soluble QD nanoparticles include cadmium-free QD nanoparticles comprising semiconductor materials selected from the group of materials consisting of ZnS, ZnSe, ZnTe, InP, InAs, InSb, AlP, AlS, AlAs, AlSb, GaN, GaP, GaAs, GaSb, PbS, PbSe, AgInS₂, AgInS₂/ZnS, Si, Ge and alloys and doped derivatives thereof. The nanoparticle may comprise a core formed of one of the materials and one or more shells of another of the materials or may be core/multi-shell QDs. In some aspects the surface modified, water soluble QD nanoparticle comprise a ligand interactive agent and a surface modifying ligand. In some embodiments the surface modified cadmium-free QD nanoparticle is formed by chemical addition of the ligand interactive agent and the surface modifying ligand to the QD in a solution comprising hexamethoxymethylmelamine. In certain embodiment the solution comprises a non-polar solvent. In particular embodiments, the non-polar solvent is toluene. In certain embodiments the ligand interactive agent is a C₈₋₂₀ fatty acid or esters thereof. The surface modifying ligand is a monomethoxy polyethylene oxide in certain aspects.

Also provided are methods of simultaneously inducing cell death, tumor shrinkage, and fluorescent labelling of tumor cells by administering a nanoparticle conjugate comprising a non-toxic water soluble QD nanoparticle that is chemically conjugated to a monoclonal antibody that is directed to an external domain of cell surface receptor that is over-expressed on the tumor cell. In certain aspects the cell surface receptor is an epidermal growth factor receptor (EGFR) and the tumor is a pancreatic tumor.

Further provided are methods of detecting or therapeutically modulating biological moieties including allowing a composition comprising target specific derivatized cadmium-free water soluble QDs to interact physically with a biological target, wherein the cadmium-free water soluble quantum dots have been surface modified using the melamine cross-linker hexamethoxymethylmelamine (HMMM) to include one or more functional groups selected from the group consisting of COOH, NH₂, SH, OH, sulfonate, phosphate, azide, allyl, silyl and PEG chains and are further derivatized via the one or more functional groups with a plurality of different target specific ligands; stimulating emission from the target specific derivatized cadmium-free water soluble QDs via application of light at an excitatory wavelength for the QD; and recording and/or imaging a spectral emission of interactions between the composition and the biological target. Therapeutic modulation by the target specific derivatized cadmium-free water soluble QDs may be provided by the spectral emission from the QD including through the generation of singlet oxygen and/or heat.

In certain aspects the ligands are selected from one or more of the group consisting of antibodies, streptavidin, nucleic acids, lipids, saccharides, drug molecules, proteins, peptides, and amino acids. In certain aspects the detecting is used for imaging and detecting one or more of angiogenesis, tumor demarcation, tumor metastasis, diagnostics in vivo, and lymph node progression while in other aspects the detecting is used in one or more of immunochemistry, immunofluorescence, DNA sequence analysis, fluorescence resonance energy transfer, flow cytometry, fluorescence activated cell sorting, and high-throughput screening. In certain aspects at least one of the ligands has specificity for a target selected from the group consisting of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand. In particular aspects a multi-ligand nano-device is provided having at least one target specific antibody ligand and a further ligand selected from streptavidin, nucleic acids, lipids, saccharides, drug molecules, proteins, peptides, and amino acids where the antibody ligand acts as a targeting ligand to deliver the further ligand.

In certain embodiments a multi-ligand nano-device is provided comprising cadmium-free water soluble QDs derivatized with at least two populations of ligands, each population having a different specificity. In certain aspects, the multi-ligands include at least one target specific antibody ligand and a further ligand selected from streptavidin, nucleic acids, lipids, saccharides, drug molecules, proteins, peptides, and amino acids where the antibody ligand acts as a targeting ligand to deliver the further ligand. In certain aspects, the multi-ligand nano-device includes at least two target specific antibody ligands at least one of which is a ligand having specificity for a target selected from the group consisting of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

In certain embodiments the multi-ligand nano-device is manufactured for use as a medicament for detecting and treating cancer, wherein device is utilized in preoperative or intraoperative diagnosis with minimally invasive endoscopy or laproscopy. In particular embodiments the multi-ligand nano-device is adapted for injection into the circulation or into the abdomen and concentration in tumor cells relative to normal cells. In one embodiment, the multi-ligand nano-device is adapted for concentration in tumor cells relative to normal cells by surface modification with monomethoxy polyethylene oxide. The concentrated multi-ligand nano-device can be induced to fluoresce by exposure to a QD excitatory light source applied endoscopically or laparoscopically, said fluorescence detectable by an imaging camera. In addition to detection via the induced fluorescence, the fluorescence may be utilized to provide therapeutic modulation by the spectral emission from the QD including through the generation of singlet oxygen and/or heat.

### BRIEF DESCRIPTION OF THE DRAWINGS

For a more complete understanding of the present invention, including features and advantages, reference is now made to the detailed description of the invention along with the accompanying figures:

FIG. 1A shows a transmission electron microscope (TEM) image (scale bar corresponds to 5 nm) and FIG. 1B shows size distribution of the resulting nanoparticles from TEM. FIG. 1C shows the hydrodynamic size of surface-treated water soluble nanoparticles using dynamic light scattering (DLS). FIG. 1D shows the photoluminescence emission spectrum of an aqueous solution of water soluble nanoparticles (1 mM) using excitation at 405 nm.

FIG. 2 depicts an embodiment of a process for surface modification of cadmium-free QDs.

FIG. 3 depicts an embodiment of a process for generation of a SAV conjugate with a surface modified water soluble QD.

FIG. 4A a schematic for a bi-ligand nano-device and mechanisms of action on cancer cells. FIG. 4B presents a schematic for an anti-EGFR and anti-PD-L1 nano-device and mechanisms of action on cancer cells according to one embodiment disclosed herein.

FIG. 5 represents a demonstration of the ease of visualization of in vivo compatible water dispersible cadmium-free QD nanoparticles in vivo. 30 μg of red in vivo compatible water dispersible cadmium-free QD nanoparticles were injected subcutaneously into the paw of a nude mouse under anesthesia. The QD nanoparticles (untargeted) migrated to the axillary lymph node (the bright spot) and were easily detected by a simple fluorescence imaging system.

FIGS. 6A and 6B demonstrate red in vivo compatible water dispersible cadmium-free QD nanoparticles were conjugated to the anti-HER2 mAb (Trastuzumab, HERCEPTIN®). In FIG. 6A the red in vivo compatible water dispersible cadmium-free QD nanoparticles conjugated to anti-HER2 mAb were used to treat HER2 positive 4T1 cells. FIG. 6B shows the same treatment with control Her2 negative 4T1 cells.

FIG. 7 demonstrates successful development of QD-streptavidin conjugates. In this case both red and green in vivo compatible water dispersible cadmium-free QD nanoparticles were conjugated to streptavidin and used to stain biotinylated polymer spheres.

FIGS. 8A and 8B demonstrate the results of experiments were undertaken to insure that the in vivo compatible water dispersible cadmium-free QD do not bind non-specifically. FIG. 8A shows the results of adding differing concentrations of HERCEPTIN® QD conjugates to the strips previously dotted with purified HER-2. FIG. 8B show the results of adding differing concentrations of the unfunctionalized in vivo compatible water dispersible cadmium-free QD to the strips previously dotted with purified HER-2.

FIG. 9 shows the results of a dilution series of HER-2 dotted on a dot blot strip followed by detection with a HERCEPTIN® QD conjugate.

FIGS. 10A and 10B show the results of binding in vivo compatible water dispersible cadmium-free QD to a SK-BR-3 cell line with and without a targeting moiety. FIG. 10A shows the results of treating SK-BR-3 cells with unfunctionalized in vivo compatible water dispersible cadmium-free QD. FIG. 10B shows the results of treating SK-BR-3 cells with trastuzumab functionalized in vivo compatible water dispersible cadmium-free QD.

### DETAILED DESCRIPTION OF THE INVENTION

The recent emergence of functional nanotechnology and molecular therapy is paving the way for novel and more efficient medical approaches. Provided herein are embodiments that provide fluorescent quantum dot nanoparticles (QDs) that feature high safety and biocompatibility profiles conjugated to tumor targeting moieties wherein the conjugate has both therapeutic and diagnostic uses. QDs are fluorescent semiconductor nanoparticles with superb optical properties. They shine around 20 times brighter and are many times more photo-stable than any of the conventional fluorescent dyes (like indocyanine green (ICG)). Importantly, QD residence times are longer due to their chemical nature and nano-size. QD can absorb and emit much stronger light intensities thus improving ease of detection. QDs also offer tunability of emission so that spatial imaging is enabled with minimum background from autofluorescence interference. Another advantage is that unlike small dyes, each QD can be equipped with more than one binding tag, forming multi-specific nano-devices such as, without limitation, bi- or tri-specific nano-devices with maximum binding probability, and thus highest detection efficiency.

The unique properties of QDs enable several medical applications that serve unmet needs in in vitro and in vivo diagnostics, clinical imaging, targeted drug delivery, and photodynamic therapy. The conjugates are thus “theranostic” nano-devices with multimodal properties useful for the imaging and treatment of cancer. In certain embodiments the disclosed theranostic nano-devices have imaging and therapeutic capabilities to be used for pre-, intra-, and post-operative detection and therapy of cancer. These devices are particularly suitable for use in the treatment of cancers for which surgical resection currently provides the only promise of a cure. Certain cancers such as adenocarcinoma are relatively slowly growing and thus are resistant to chemotherapeutic and radiotherapies that target rapidly dividing cells. A potentially curative approach is to surgically remove the adenocarcinoma before it becomes metastatic. Problematically, certain cancers, such as adenocarcinoma of the exocrine pancreas, are often metastatic upon diagnosis. Coupled with this, tumorous tissue is difficult to discriminate from normal tissue during resection thus leaving tumor tissue in place.

Based in part on staging difficulties, the presumed dissemination in many patients, and the difficulty of the resection procedure, currently only 8% of UK pancreatic cancer patients receive surgical resection. Presently, the 10-year survival rate for pancreatic cancer is <1%. What is critically needed in the treatment of pancreatic cancer is the ability to intraoperatively identify and remove cancerous tissue including small metastases.

Efforts to provide in vivo labelling and identification of tumor cells sufficient to support adequate resection has been undertaken but small molecule dyes, organic dyes and carbon black inks lack specificity and tend to quickly stain all the surroundings. Recently, fluorescence imaging using organic dyes (fluorescein or indocyanine green (ICG)) has been introduced and, while fluorescent dyes can improve selectivity, they are limited by their rapid clearance, fast fading, fast metabolic degradation and low signal intensity. See e.g. Condeelis J and Weissleder R. In Vivo Imaging in Cancer. Cold Spring Harb Perspect Biol. 2010, 2:a003848. The present inventors appreciated that an ideal intraoperative diagnostic tool for supporting more complete resection would also provide a direct tumorocidal modality for remaining cells.

Appreciating the shortcomings of existing technologies, the present inventors undertook to provide theranostic nano-devices that are selective to target molecules that are over-expressed in cancer cells with initial focus on pancreatic cancer cells. One such target molecule is Epidermal Growth Factor Receptor (EGFR). EGFR, a.k.a. HER1, is a cell surface molecule that is a member of the erbB receptor tyrosine kinase family. Following binding of EGF, EGFR homodimerizes (or heterodimerizes with other erbB family members) and signaling is initiated through the phosphoinositide 3-kinase (PI-3K)/AKT and mitogen-activated protein (MAP) kinase pathways. Cell signaling mediated through EGFR/HER1 promotes tumor proliferation, angiogenesis, metastasis, and evasion of apoptosis. EGFR is over-expressed in several malignancies, including head and neck, renal, colorectal, lung, breast, prostate and pancreatic cancers. Because EGFR is a promising target for cancer therapy, monoclonal antibodies (mAb) have been developed that bind to EGFR and block binding of the EGF ligand. One such mAb is the human-mouse chimeric antibody cetuximab (marketed by Eli Lilly under the tradename ERBITUX®). Cetuximab is FDA approved for the treatment of EGFR-positive colorectal cancer and certain types of head and neck cancer together with particular chemotherapeutic regimens.

EGFR is also an identified target for diagnosis and treatment of pancreatic cancer. EGFR is over-expressed by pancreatic tumor cells in about 70% of patients and such over-expression has been associated with a poor prognosis. On this basis, targeting EGFR through the use of cetuximab has been studied in pancreatic cancer. Unfortunately, phase II and phase III trials have failed to consistently show efficacy of cetuximab treatment in advanced pancreatic cancer either alone or in combination with cytotoxic agents. See Luedke, E., et al. Monoclonal Antibody Therapy of Pancreatic Cancer with Cetuximab: Potential for Immune Modulation. J Immunother. 35 (5) (2012) 367-373. In addition to administration of cetuximab alone, use of cetuximab conjugates has been attempted. To such end, cetuximab has been used as a targeting agent to deliver ˜5 nm gold nanoparticles that are coderivatized with the cytotoxic agent gemcitabine. See Patra, C R, et al. Targeted Delivery of Gemcitabine to Pancreatic Adenocarcinoma: Using Cetuximab as a Targeting Agent. Cancer Res 68(6) (2008) 1970-1978. In the Patra study, the gold nanoparticles were used as a non-toxic derivatizable vehicle that is easily synthesized and characterized. However, the gold particle carriers have no active diagnostic properties and biodistribution of the gold nanoparticles must be done post-mortem obviating the possibility of intraoperative use.

In certain examples provided herein, the target cancer is pancreatic cancer because it is often lethal and therefore effective diagnostic and therapeutic strategies are urgently needed to improve survival rates. In addition, staging and precision surgery of pancreatic tumor lesions are challenging due to their soft and unresectable nature. In certain embodiments, the nano-device is engineered as a conjugate of biocompatible, non-toxic, fluorescent quantum dot nanoparticles (QDs) with anti-EGFR mAb. In other embodiments, a conjugate of biocompatible, non-toxic, fluorescent QDs with an anti-PD-L1 checkpoint mAb is provided. In still further embodiments, multivalent QD nanoparticles are provided with combined specificity against EGFR and PD-L1. The concepts exemplified using anti-EGFR and PD-L1 are applicable to targeting other types of cancers by using the relevant mAbs or their derivatives.

In one embodiment, non-toxic water soluble QDs are chemically attached to an antibody directed to the extracellular domain of the human epidermal growth factor receptor (EGFR). Cetuximab is one such antibody that is presently FDA approved and indicated for the treatment of locally or regionally advanced squamous cell carcinoma of the head and neck, colorectal, and lung cancers. Many studies are also considering the use of cetuximab in pancreatic cancer and many other cancers that have high expression of EGFR.

Thus, in one embodiment QDs are synthesized that are non-toxic and water soluble (biocompatible) and are surface equipped with a conjugation capable function (COOH, OH, NH₂, SH, azide, alkyne). In one exemplified embodiment, the water soluble non-toxic QD is or becomes carboxyl functionalized. The COOH-QD is linked to the amine terminus of a tumor targeting antibody such as cetuximab using a carbodiimide linking technology employing water-soluble 1-ethyl-3-(-3-dimethylaminopropyl) carbodiimide hydrochloride (EDC). The carboxyl functionalized QD is mixed with EDC to form an active O-acylisourea intermediate that is then displaced by nucleophilic attack from primary amino groups on the monoclonal antibody in the reaction mixture. If desired, a sulfo derivative of N-hydroxysuccinimide (sulfo-NHS) is added during the reaction with the primary amine bearing antibody. With the sulfo-NHS addition, the EDC couples NHS to carboxyls, forming an NHS ester that is more stable than the O-acylisourea intermediate while allowing for efficient conjugation to primary amines at physiologic pH. In either event, the result is a covalent bond between the QD and the antibody. Other chemistries like Suzuki-Miyaura cross-coupling (SMCC), or aldehyde based reactions may alternatively be used.

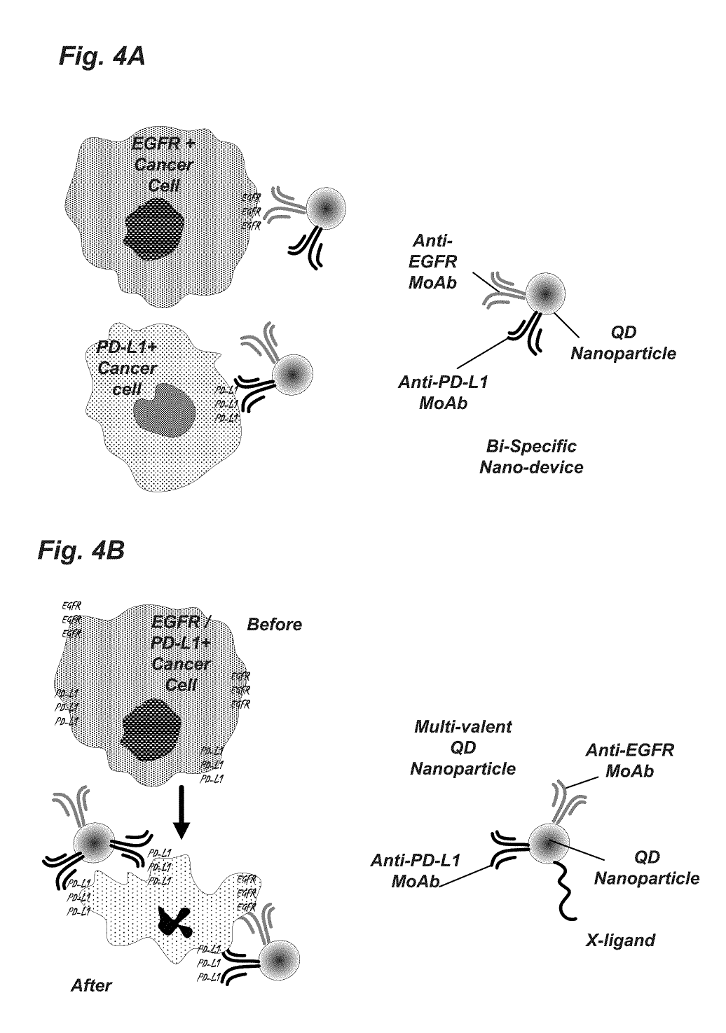

As shown in FIGS. 4A and B, the rationale of this embodiment is to take advantage of the ability of an anti-EGFR mAb such as cetuximab to specifically bind and inhibit EGFR on tumors and to use it as a specific label by conjugation to fluorescent QDs. The formed (QD-Cetuximab) agent carries four different functionalities:
Tumor labeling for identification of tumor tissue intraoperatively due to the fluorescence properties of the QDs,
EGFR inhibition due to cetuximab binding to EGFR,
Synergized tumor uptake due to EPR (Enhanced Permeability and Retention) effect contributed by the nano size of the conjugate,
Tumor phototherapeutic effect due to the ability of the QDs to generate singlet oxygen or condense energy in the form of heat that can trigger cell suicidal cascade (apoptosis).

The combination of the above four mechanisms are believed to provide synergistic effects, which could provide a very efficient treatment for EGFR-expressing cancers and with lower side effects than with cetuximab treatment alone.

While the making and using of various embodiments of the present invention are discussed in detail below, it should be appreciated that the present invention provides many applicable inventive concepts which can be employed in a wide variety of specific contexts. The specific embodiments discussed herein are merely illustrative of specific ways to make and use the invention and do not delimit the scope of the invention.

### Abbreviations

The following abbreviations are used throughout this application:
CA125 Cancer Antigen-125 (CA125) is also known as mucin 16 (MUC16)
CA19-9 Carbohydrate antigen 19-9 (CA19-9), a.k.a. cancer antigen 19-9 or sialylated Lewis (a) antigen
CD20 CD20 is an activated-glycosylated phosphoprotein encoded by the MS4A1 gene and expressed on the surface of B-cells but not early pro-B cells or plasma cells.
CD25 CD25 is the interleukin-2 receptor alpha chain (IL2RA) protein
CD30 a.k.a. TNFRSF8 is a tumor necrosis factor receptor family protein
CD33 a.k.a. sialic acid binding Ig-like lectin 3 (SIGLEC-3)
CD52 Cluster of Differentiation 52, a.k.a. Cambridge Pathology Antigen-1 (Campath-1 antigen)
CD73 Cluster of Differentiation 73, a.k.a. ecto-5′-nucleotidase (NT5E)
CD109 Cluster of Differentiation 109, is a glycosyl phosphatidylinositol (GPI)—linked glycoprotein that is involved in transforming growth factor beta binding
CEA Carcinoembryonic Antigens
CTLA-4 Cytotoxic T-lymphocyte-associated protein 4
EGFR Epidermal Growth Factor Receptor, a.k.a. epidermal growth factor receptor (EGFR, or HER1) belongs to the erbB receptor tyrosine kinase family.
HER2 Human Epidermal Growth Factor Receptor 2
PD-L1 Programmed death ligand-1 (PD-L1), also known as cluster of differentiation 274 (CD274) or B7 homolog 1 (B7-H1).
QD Quantum Dot
QY Quantum Yield
RANK Receptor Activator of Nuclear Factor Kappa-B
SLN Sentinel Lymph Node
VEGF-A Vascular Endothelial Growth Factor A

(RANK) Ligand (or RANKL) is also known as tumor necrosis factor ligand superfamily member 11 (TNFSF11), TNF-related activation-induced cytokine (TRANCE), osteoprotegerin ligand (OPGL), and osteoclast differentiation factor (ODF).

The terms “comprising” (and any form thereof such as “comprise” and “comprises”), “having” (and any form thereof such as “have” and “has”), “including” (and any form thereof such as “includes” and “include”) or “containing” (and any form thereof such as “contains” and “contain”) are inclusive or open-ended and do not exclude additional, unrecited elements or method steps.

As used herein the term “antibody” includes both intact immunoglobulin molecules as well as portions, fragments, and derivatives thereof, such as, for example, Fab, Fab′, F(ab′)₂, Fv, Fsc, CDR regions, or any portion of an antibody that is capable of binding an antigen or epitope including chimeric antibodies that are bi-specific or that combine an antigen binding domain originating with an antibody with another type of polypeptide. The term antibody thus includes monoclonal antibodies (mAb), chimeric antibodies, humanized antibodies, as well as fragments, portions, regions, or derivatives thereof, provided by any known technique including but not limited to, enzymatic cleavage and recombinant techniques. The term “antibody” as used herein also includes single-domain antibodies (sdAb) and fragments thereof that have a single monomeric variable antibody domain (V_{H}) of a heavy-chain antibody. sdAb, which lack variable light (V_{L}) and constant light (C_{L}) chain domains are natively found in camelids (V_{H}H) and cartilaginous fish (V_{NAR}) and are sometimes referred to as “Nanobodies” by the pharmaceutical company Ablynx who originally developed specific antigen binding sdAb in llamas. The modifier “monoclonal” indicates the character of the antibody as being obtained from a substantially homogeneous population of antibodies, and is not to be construed as requiring production of the antibody by any particular method.

As used herein the compound “guaifenesin” has the following chemical structure:

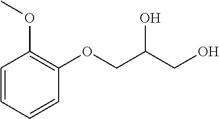

As used herein the compound “salicylic acid” has the following chemical structure:

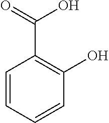

Methods of synthesizing core and core-shell nanoparticles are disclosed, for example, in co-owned U.S. Pat. Nos. 7,867,556, 7,867,557, 7,803,423, 7,588,828, and 6,379,635. The contents of each of the forgoing patents are hereby incorporated by reference, in their entirety. U.S. Pat. Nos. 9,115,097, 8,062,703, 7,985,446, 7,803,423, and 7,588,828, and U.S. Publication Nos. 2010/0283005, 2014/0264196, 2014/0277297 and 2014/0370690, the entire contents of each of which are hereby incorporated by reference, describe methods of producing large volumes of high quality monodisperse QDs.

A nanoparticle's compatibility with a medium as well as the nanoparticle's susceptibility to agglomeration, photo-oxidation and/or quenching, is mediated largely by the surface composition of the nanoparticle. The coordination about the final inorganic surface atoms in any core, core-shell or core/multi-shell nanoparticle may be incomplete, with highly reactive “dangling bonds” on the surface, which can lead to particle agglomeration. This problem is overcome by passivating (capping) the “bare” surface atoms with protecting organic groups, referred to herein as capping ligands or a capping agent. The capping or passivating of particles prevents particle agglomeration from occurring but also protects the particle from its surrounding chemical environment and provides electronic stabilization (passivation) to the particles, in the case of core material. The capping ligand is usually a Lewis base bound to surface metal atoms of the outer most inorganic layer of the particle. The nature of the capping ligand largely determines the compatibility of the nanoparticle with a particular medium.

In many QD materials, the capping ligands are hydrophobic (for example, alkyl thiols, fatty acids, alkyl phosphines, alkyl phosphine oxides, and the like). Thus, the nanoparticles are typically dispersed in hydrophobic solvents, such as toluene, following synthesis and isolation of the nanoparticles. Such capped nanoparticles are typically not dispersible in more polar media. If surface modification of the QD is desired, the most widely used procedure is known as ligand exchange. Lipophilic ligand molecules that coordinate to the surface of the nanoparticle during core synthesis and/or shelling procedures may subsequently be exchanged with a polar/charged ligand compound. An alternative surface modification strategy intercalates polar/charged molecules or polymer molecules with the ligand molecules that are already coordinated to the surface of the nanoparticle. However, while certain ligand exchange and intercalation procedures render the nanoparticle more compatible with aqueous media, they may result in materials of lower photoluminescence quantum yield (QY) and/or substantially larger size than the corresponding unmodified nanoparticle. Problematically, for the theranostic purposes disclosed herein, the QD is preferably substantially free of toxic heavy metals such as cadmium, lead and arsenic (e.g., contains less than 5 wt. %, such as less than 4 wt. %, less than 3 wt. %, less than 2 wt. %, less than 1 wt. %, less than 0.5 wt. %, less than 0.1 wt. %, less than 0.05 wt. %, or less than 0.01 wt. % of heavy metals such as cadmium, lead and arsenic) or is free of heavy metals such as cadmium, lead and arsenic. Examples of cadmium, lead and arsenic free nanoparticles include nanoparticles comprising semiconductor materials, e.g., ZnS, ZnSe, ZnTe, InP, InAs, InSb, AlP, AlS, AlAs, AlSb, GaN, GaP, GaAs, GaSb, PbS, PbSe, AgInS₂, AgInS₂/ZnS, Si, Ge, and alloys and doped derivatives thereof, particularly, nanoparticles comprising cores of one of these materials and one or more shells of another of these materials.

It is noted that nanoparticles that include a single semiconductor material, e.g., CdS, CdSe, ZnS, ZnSe, InP, GaN, etc. may have relatively low QY because of non-radiative electron-hole recombination that occurs at defects and dangling bonds at the surface of the nanoparticles. In order to at least partially address these issues, the nanoparticle cores may be at least partially coated with one or more layers (also referred to herein as “shells”) of a material different than that of the core, for example a different semiconductor material than that of the “core.” The material included in the one or more shells may incorporate ions from any one or more of groups 2 to 16 of the periodic table. When a nanoparticle has two or more shells, each shell may be formed of a different material. In an exemplary core/shell material, the core is formed from one of the materials specified above and the shell includes a semiconductor material of larger band-gap energy and similar lattice dimensions as the core material. Exemplary shell materials include, but are not limited to, ZnS, ZnO, MgS, MgSe, MgTe and GaN. One example of a multi-shell nanoparticle is InP/ZnS/ZnO. The confinement of charge carriers within the core and away from surface states provides nanoparticles of greater stability and higher QY.

However, while it is desirable to have QD that lack toxic heavy metals, it has proved particularly difficult to modify the surface of cadmium-free QDs. Cadmium-free QDs readily degrade when methods such as the aforementioned ligand exchange methods are used to modify the surface of such cadmium-free QDs. For example, attempts to modify the surface of cadmium-free QDs have been observed to cause a significant decrease in the QY of such nanoparticles. For the in vivo purposes disclosed herein, surface-modified cadmium-free QDs with high QY are required. For purposes of the present invention, when referring to water dispersible cadmium-free QDs: QY of <20% are considered very low; QY of <30% are considered low; QY of 30-40% are considered medium; QY>40% are considered high and QY>50% are considered very high.

The high QY cadmium-free water dispersible QDs disclosed herein have a QY greater than about 40%. For certain in vivo embodiments, heavy metal-free semi-conductor indium-based QDs or QDs containing indium and/or phosphorus are preferred.

In certain embodiments, non-toxic QD nanoparticles are surface modified to enable them to be water soluble and to have surface moieties that allow derivatization by exposing them to a ligand interactive agent to effect the association of the ligand interactive agent and the surface of the QD. The ligand interactive agent can comprise a chain portion and a functional group having a specific affinity for, or reactivity with, a linking/crosslinking agent, as described below. The chain portion may be, for example, an alkane chain. Examples of functional groups include nucleophiles such as thio groups, hydroxyl groups, carboxamide groups, ester groups, and a carboxyl groups. The ligand interactive agent may, or may not, also comprise a moiety having an affinity for the surface of a QD. Examples of such moieties include thiols, amines, carboxylic groups, and phosphines. If ligand interactive group does not comprise such a moiety, the ligand interactive group can associate with the surface of nanoparticle by intercalating with capping ligands. Examples of ligand interactive agents include C₈₋₂₀ fatty acids and esters thereof, such as for example isopropyl myristate.

It should be noted that the ligand interactive agent may be associated with QD nanoparticle simply as a result of the processes used for the synthesis of the nanoparticle, obviating the need to expose nanoparticle to additional amounts of ligand interactive agents. In such case, there may be no need to associate further ligand interactive agents with the nanoparticle. Alternatively, or in addition, QD nanoparticle may be exposed to ligand interactive agent after the nanoparticle is synthesized and isolated. For example, the nanoparticle may be incubated in a solution containing the ligand interactive agent for a period of time. Such incubation, or a portion of the incubation period, may be at an elevated temperature to facilitate association of the ligand interactive agent with the surface of the nanoparticle. Following association of the ligand interactive agent with the surface of nanoparticle, the QD nanoparticle is exposed to a linking/crosslinking agent and a surface modifying ligand. The linking/crosslinking agent includes functional groups having specific affinity for groups of the ligand interactive agent and with the surface modifying ligand. The ligand interactive agent-nanoparticle association complex can be exposed to linking/crosslinking agent and surface modifying ligand sequentially. For example, the nanoparticle might be exposed to the linking/crosslinking agent for a period of time to effect crosslinking, and then subsequently exposed to the surface modifying ligand to incorporate it into the ligand shell of the nanoparticle. Alternatively, the nanoparticle may be exposed to a mixture of the linking/crosslinking agent and the surface modifying ligand thus effecting crosslinking and incorporating surface modifying ligand in a single step.

The following examples are include for the sake of completeness of disclosure and to illustrate the methods of making the compositions and composites of the present invention as well as to present certain characteristics of the compositions. In no way are these examples intended to limit the scope or teaching of this disclosure.

### Example 1

### Synthesis of Non-Toxic Quantum Dots

A molecular seeding process was used to generate non-toxic QDs. Briefly, the preparation of non-functionalized indium-based QDs with emission in the range of 500-700 nm was carried out as follows: Dibutyl ester (approximately 100 ml) and myristic acid (MA) (10.06 g) were placed in a three-neck flask and degassed at ˜70° C. under vacuum for 1 h. After this period, nitrogen was introduced and the temperature was increased to ˜90° C. Approximately 4.7 g of the ZnS molecular cluster [Et₃NH]₄ [Zn₁₀S₄(SPh)₁₆] was added, and the mixture was stirred for approximately 45 min. The temperature was then increased to ˜100° C., followed by the drop-wise additions of In(MA)₃ (1M, 15 ml) followed by trimethylsilyl phosphine (TMS)₃P (1M, 15 ml). The reaction mixture was stirred while the temperature was increased to ˜140° C. At 140° C., further drop-wise additions of indium myristate (In(MA)₃) dissolved in di-n-butylsebacate ester (1M, 35 ml) (left to stir for 5 min) and (TMS)₃P dissolved in di-n-butylsebacate ester (1M, 35 ml) were made. The temperature was then slowly increased to 180° C., and further dropwise additions of In(MA)₃ (1M, 55 ml) followed by (TMS)₃P (1M, 40 ml) were made. By addition of the precursor in this manner, indium-based particles with an emission maximum gradually increasing from 500 nm to 720 nm were formed. The reaction was stopped when the desired emission maximum was obtained and left to stir at the reaction temperature for half an hour. After this period, the mixture was left to anneal for up to approximately 4 days (at a temperature ˜20-40° C. below that of the reaction). A UV lamp was also used at this stage to aid in annealing.

The particles were isolated by the addition of dried degassed methanol (approximately 200 ml) via cannula techniques. The precipitate was allowed to settle and then methanol was removed via cannula with the aid of a filter stick. Dried degassed chloroform (approximately 10 ml) was added to wash the solid. The solid was left to dry under vacuum for 1 day. This procedure resulted in the formation of indium-based nanoparticles on ZnS molecular clusters. In further treatments, the quantum yields of the resulting indium-based nanoparticles were further increased by washing in dilute hydrofluoric acid (HF). The quantum yields of the indium based core material ranged from approximately 25%-50%.

Growth of a ZnS shell: A 20 ml portion of the HF-etched indium-based core particles was dried in a three-neck flask. 1.3 g of myristic acid and 20 ml di-n-butyl sebacate ester were added and degassed for 30 min. The solution was heated to 200° C., and 2 ml of 1 M (TMS)₂S was added drop-wise (at a rate of 7.93 ml/h). After this addition was complete, the solution was left to stand for 2 min, and then 1.2 g of anhydrous zinc acetate was added. The solution was kept at 200° C. for 1 hr and then cooled to room temperature. The resulting particles were isolated by adding 40 ml of anhydrous degassed methanol and centrifuging. The supernatant liquid was discarded, and 30 ml of anhydrous degassed hexane was added to the remaining solid. The solution was allowed to settle for 5 h and then centrifuged again. The supernatant liquid was collected and the remaining solid was discarded. The quantum yields of the final non-functionalized indium-based QD nanoparticle material ranged from approximately 60%-90% in organic solvents.

### Example 2

### Water Soluble Surface Modified QDs

Provided herein is one embodiment of a method for generating and using melamine hexamethoxymethylmelamine (HMMM) modified fluorescent nanoparticles as nano probes to detect and/or modulate biological targets in vitro and in vivo. The unique melamine-based coating presents excellent biocompatibility, low toxicity and very low non-specific binding. These unique features allow a wide range of biomedical applications both in vitro and in vivo.

One example of preparation of a suitable water soluble QD is provided as follows: 200 mg of cadmium-free QD nanoparticles with red emission at 608 nm having as a core material an alloy comprising indium and phosphorus with Zn containing shells as described in Example 1 was dispersed in toluene (1 ml) with isopropyl myristate (100 microliters). The isopropyl myristate is included as the ligand interactive agent. The mixture was heated at 50° C. for about 1-2 minutes then slowly shaken for 15 hours at room temperature. A toluene solution (4 ml) of hexamethoxymethylmelamine (HMMM) (CYMEL 303, available from Cytec Industries, Inc., West Paterson, N.J.) (400 mg), monomethoxy polyethylene oxide (CH₃O-PEG₂₀₀₀-OH) (400 mg), and salicylic acid (50 mg) was added to the nanoparticle dispersion. The salicylic acid that is included in the functionalization reaction plays three roles, as a catalyst, a crosslinker, and a source for COOH. Due in part to the preference of HMMM for OH groups, many COOH groups provided by the salicylic acid remain available on the QD after crosslinking.

HMMM is a melamine-based linking/crosslinking agent having the following structure:

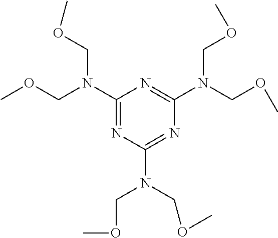

HMMM can react in an acid-catalyzed reaction to crosslink various functional groups, such as amides, carboxyl groups, hydroxyl groups, and thiols.

The mixture was degassed and refluxed at 130° C. for the first hour followed by 140° C. for 3 hours while stirring at 300 rpm with a magnetic stirrer. During the first hour a stream of nitrogen was passed through the flask to ensure the removal of volatile byproducts generated by the reaction of HMMM with nucleophiles. The mixture was allowed to cool to room temperature and stored under inert gas. The surface-modified nanoparticles showed little or no loss in fluorescence quantum yield and no change in the emission peak or full width at half maximum (FWHM) value, compared to unmodified nanoparticles. An aliquot of the surface-modified nanoparticles was dried under vacuum and deionized water was added to the residue. The surface modified nanoparticles dispersed well in the aqueous media and remained dispersed permanently. In contrast, unmodified nanoparticles could not be suspended in the aqueous medium. The fluorescence QY of the surface-modified nanoparticles according to the above procedure is approximately 47.

In another embodiment, cadmium-free quantum dot nanoparticles (200 mg) with red emission at 608 nm were dispersed in toluene (1 ml) with cholesterol (71.5 mg). The mixture was heated at 50° C. for about 1-2 minutes then slowly shaken for 15 hours at room temperature. A toluene solution (4 ml) of HMMM (Cymel 303) (400 mg), monomethoxy polyethylene oxide (CH₃O-PEG₂₀₀₀-OH) (400 mg), guaifenesin (100 mg), dichloromethane (DCM) (2 mL) and salicylic acid (50 mg) was added to the nanoparticle dispersion. The mixture was degassed and refluxed at 140° C. for 4 hours while stirring at 300 rpm with a magnetic stirrer. As with the prior procedure, during the first hour a stream of nitrogen was passed through the flask to ensure the removal of volatile byproducts generated by the reaction of HMMM with nucleophiles. The mixture was allowed to cool to room temperature and stored under inert gas. An aliquot of the surface-modified nanoparticles was dried under vacuum and deionized water was added to the residue. The pH of the solution was adjusted to 6.5 using a 100 mM KOH solution and the excess non reacted material was removed by three cycles of ultrafiltration using Amicon filters (30 kD). The final aqueous solution was kept refrigerated until use.

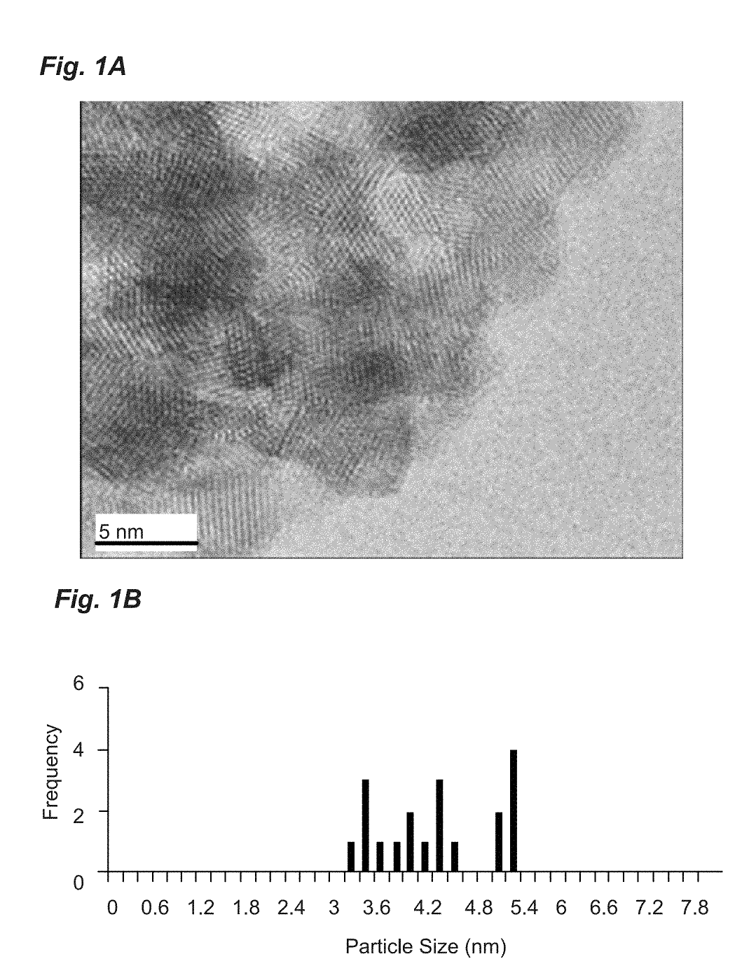

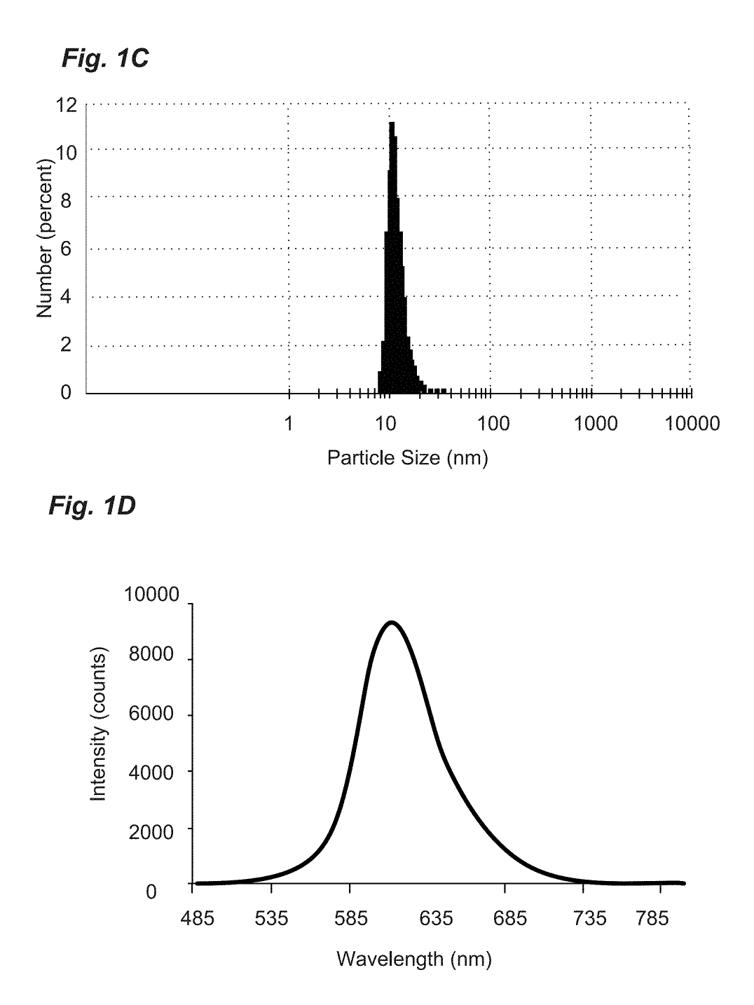

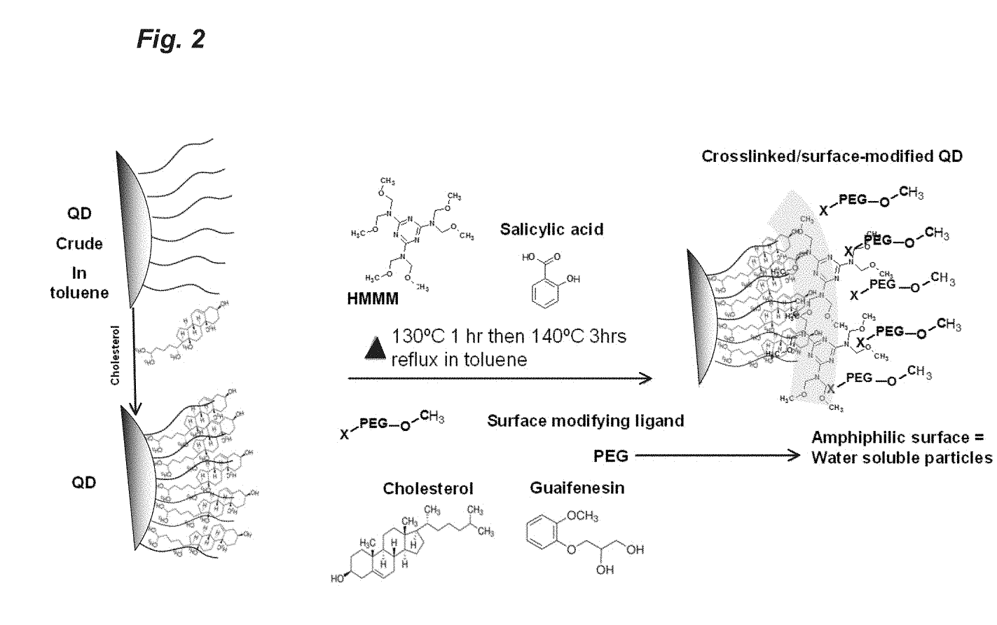

Transmission electron microscopy (TEM) analysis of the resulting nanoparticles was challenging due to the low electron density nature of the particles. FIGS. 1A and B show a typical TEM image and the size distribution of a typical sample of the nanoparticles. The TEM images were acquired using a JEOL 2010 analytical TEM. The hydrodynamic particle size was determined by the measurement of dynamic light scattering (DLS) using a Malvern Zetasizer μV system. The nanoparticles were dispersed in aqueous buffer (HEPES 6 mM, pH 7.8). The average hydrodynamic size of the surface-treated water soluble particles generated according to Example 2 is 12.2 nm with a standard deviation of 0.29 nm (FIG. 1C). FIG. 1D shows the photoluminescence emission spectrum of the surface treated nanoparticles in distilled water, which shows peak emission at 615 nm. The photoluminescence emission spectrum of QDs was recorded using a fibre optic CCD spectrometer (USB4000, Ocean Optics Inc.). For the QY measurement a spectrometer incorporating an integrating sphere was used. Quantum Yield (QY) is a well-known term in the art. It represents the number of photons emitted divided by the number of photons absorbed, expressed as a percentage. It is measured on a spectrometer incorporating an integrating sphere, relative to a dye standard. FIG. 2 depicts the generation process and resulting surface modified QD.

It is noteworthy that traditional methods for modifying nanoparticles to increase their water solubility (e.g., ligand exchange with mercapto-functionalized water soluble ligands) are ineffective under mild conditions to render the nanoparticles water soluble. Under harsher conditions, such as heat and sonication, the fraction that becomes water soluble has very low QY (<20%). The instant method, in contrast, provides water soluble nanoparticles with high or very high QY of greater than 40%. The surface-modified nanoparticles prepared as in this example also disperse well and remain permanently dispersed in other polar solvents, including ethanol, propanol, acetone, methylethylketone, butanol, tripropylmethylmethacrylate, or methylmethacrylate.

### Example 3

### QD Theranostic Devices

For in vivo purposes, QD with low toxicity profiles are desirable if not required. In one embodiment, reduced toxicity QD that lack heavy metals such as cadmium, lead and arsenic are provided for use in image-guided surgery of cancer including pancreatic, colorectal, head & neck, stomach, brain, prostate and esophageal cancers.

The unique properties of QDs enable several potential medical applications including unmet in vitro and in vivo diagnostics, clinical imaging, targeted drug delivery, and photodynamic therapy. One of the major concerns regarding the medical applications of QDs has been that the majority of research has focused on QDs containing toxic heavy metals such as cadmium, lead or arsenic. The biologically compatible and water-soluble heavy metal-free QDs described herein can safely be used in medical applications both in vitro and in vivo. In certain embodiments, in vivo compatible water dispersible cadmium-free QD are provided that have a hydrodynamic size of 10-20 nm (within the range of the dimensional size of a full IgG2 antibody). In one embodiment, the in vivo compatible water dispersible cadmium-free QD are produced in accordance with the procedures set out in Examples 1 and 2 herein. In certain embodiments, the in vivo compatible water dispersible cadmium-free QD are carboxyl functionalized and further derivatized with one or more ligand binding moieties.

In vitro and in vivo toxicology studies with the in vivo compatible water dispersible cadmium-free QD disclosed herein showed them to be are at least 20 times less cytotoxic than commercially available cadmium-based QDs, and no toxicity signs were observed on animal models at multiple times higher than useful doses. Furthermore, the in vivo compatible water dispersible cadmium-free QD nanoparticles herein provided showed no hemolytic effect and no complement C3 activation, indicating a favorable clinical compatibility profile.

Many cancers metastasize via the lymphatic system, therefore the presence of lymph node metastasis is an important prognostic marker in many cancers including melanoma, breast, colon, lung and ovarian cancers. A sentinel lymph node (SLN) is defined as the first lymph node(s) to receive lymphatic drainage from the site of a tumor. Breast cancer cells are most likely to spread to the lymph nodes (LNs) located in the axilla, therefore accurate assessment of the axillary lymph nodes (ALN) is the most prognostic indicator of survival and recurrence in patients with (early-stage) breast cancer.

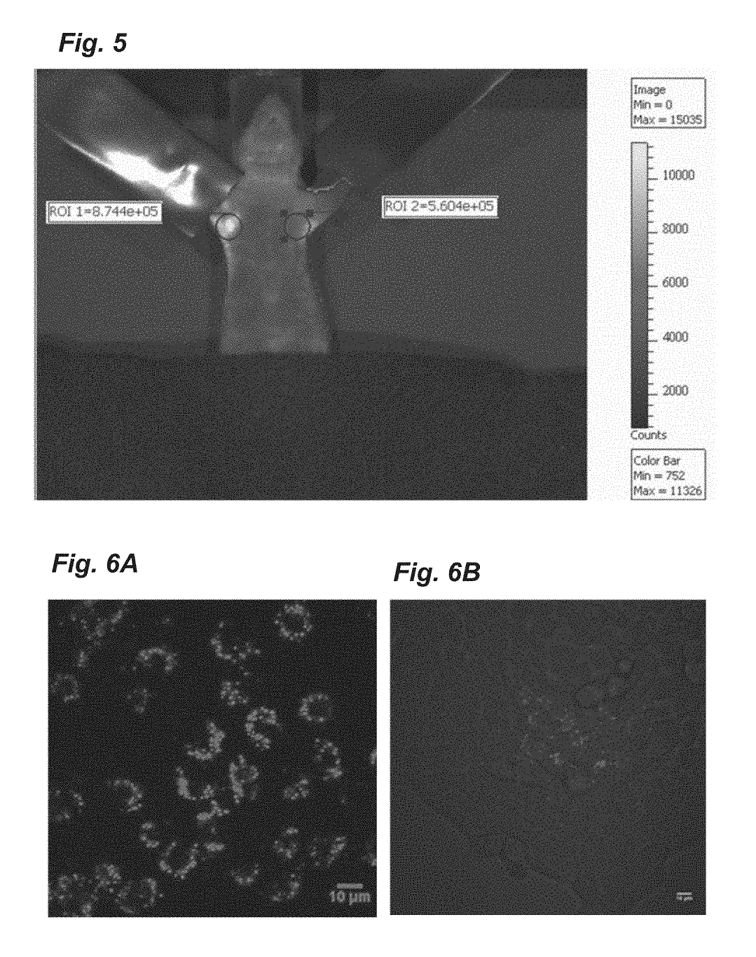

When evaluated as a marker for SLN mapping, a 30 μg dose of non-functionalized in vivo compatible water dispersible cadmium-free QD particles (passive particles) were injected subcutaneously into the paw of a nude mouse under anesthesia and were shown to be able to detect the location of the axillary lymph node using a simple imaging system. As shown in FIG. 5, heavy metal-free/cadmium-free and biocompatible QD nanoparticles can be utilized for lymph node mapping by ex vivo imaging of regional lymph nodes after subcutaneous injection. Using photoluminescence imaging and chemical extraction measurements based on elemental analysis by inductively coupled plasma mass spectroscopy, the QDs were shown to accumulate quickly and selectively in the axillary and thoracic regional lymph nodes. In addition, lifetime imaging microscopy of the QD photoluminescence indicated minimal perturbation to their photoluminescence properties in biological systems.

It has also been demonstrated that in vivo compatible water dispersible cadmium-free QD particles provided herein may be conjugated to proteins like streptavidin and the anti-HER2 monoclonal antibody Trastuzumab using carbodiimide coupling, and that the QD conjugates showed specific labelling of targeted substrates (e.g., HER2 receptor bearing cells).

Covalent Conjugation of In Vivo Compatible Water Dispersible Cadmium Free QD with Streptavidin:

The water soluble and functionalized QDs cited in Example 2 were covalently linked to streptavidin (SAV) using carbodiimide chemistry to link the surface COOH groups with the amine terminus of the SAV molecule. For example, the following protocol was used to conjugate 633 nm emissive water soluble QDs to SAV.

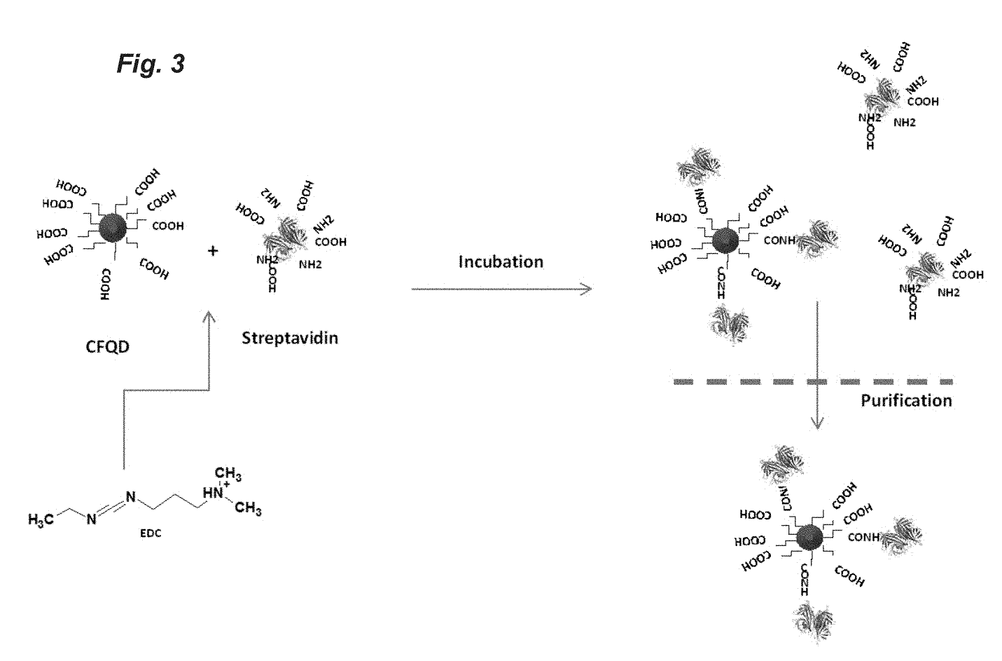

In one embodiment, in Eppendorf tubes 0.5 mg (50 μl) of water soluble nanoparticles was mixed with 100 μl MES activation buffer. The MES buffer is prepared as a 25 mM solution (2-(N-Morpholino) ethanesulfonic acid hemisodium salt (MES), Sigma Aldrich) in DI water, pH 4.0. Then 100 μl of fresh EDC (1-ethyl-3-(3-dimethylaminopropyl) carbodiimide hydrochloride, Fisher Scientific) in a 10 mg/ml solution was added, mixed well and left to stand at RT for 5 min. The mixture was transferred to pre-wetted NanoSep 300K filters, 200 μl of MES activation buffer was added, and the filtration unit was spun at 5000 rpm/20 min using a bench top micro-centrifuge (˜2000 g). The retained dots were re-dispersed in 50 μl activation buffer and transferred to an Eppendorf tube containing 0.5 mg SAV in 1×PBS. They were mixed well and incubated at room temperature for 10 min then left in the refrigerator (4-6° C.) overnight. The excess SAV was removed by three cycles of ultrafiltration using Nanosep 300K filters and PBS buffer. Each cycle of centrifugation is at 5000 rpm for 20 min and the final residue was re-dispersed with ˜400 μl PBS. The generation of the purified QD-SAV conjugate is depicted in FIG. 3.

In another embodiment, in Eppendorf tubes, 1.2 mg carboxyl-functionalised, water-soluble quantum dots were mixed with 100 μl MES activation buffer at pH 4.5 (i.e. 25 μl of a 50 mg/ml stock into 100 μl MES). To this was added 33 μl of fresh EDC solution (30 mg/ml stock in DI water, 1-ethyl-3-(3-dimethylaminopropyl) carbodiimide hydrochloride (EDC), Fisher Scientific) and the solution was mixed. To this was added 4 μl of fresh sulfo-NHS (100 mg/ml stock, ThermoFisher Scientific, in DI water) and the solution was mixed. NanoSep 300K filters (PALL NanoSep 300K Omega ultrafilters) were pre-wetted in 100 μl MES. The MES/EDC/Sulfo-NHS/QD solution was added to the NanoSep 300K filter and topped up to 500 μl with MES. The filter was centrifuged at 5000 rpm/15 min. The retained dots were re-dispersed in 50 μl activation buffer and transferred to an Eppendorf tube containing 5 μl of Streptavidin solution (SAV, Sigma Aldrich) (1 mg/100 μl stock of SAV in HEPES, 25 mM in DI water, pH 7.4)+20 μl HEPES. The solution was mixed well and incubated at room temperature overnight (around 16-18 hours).

The solution was quenched with 16 μl of a 6-amino caproic acid solution (19.7 mg/100 mM). The solution was transferred to a pre-whetted Nanosep 300K filter (100 μl 1× PBS) and topped-up to the 500 μl line with 1×PBS. Excess SAV was removed by three cycles of ultrafiltration using Nanosep 300K filters and 1×PBS buffer. Each cycle of centrifugation was 5000 rpm for 20 min with re-dispersal with ˜400 μl of 1×PBS after each cycle. The final concentrated was re-dispersed in 100 μl PBS. Activity validation was performed by mixing 10 μl of biotinylated spheres (Spherotech 10 microns) with 5 μl SAV-QD conjugate. To this was added 85 μl of 1×PBS and the mixture was left for 1 hr with shaking followed by addition of 1 ml of 1×PBS and centrifugation at 1000 rpm/5 min.

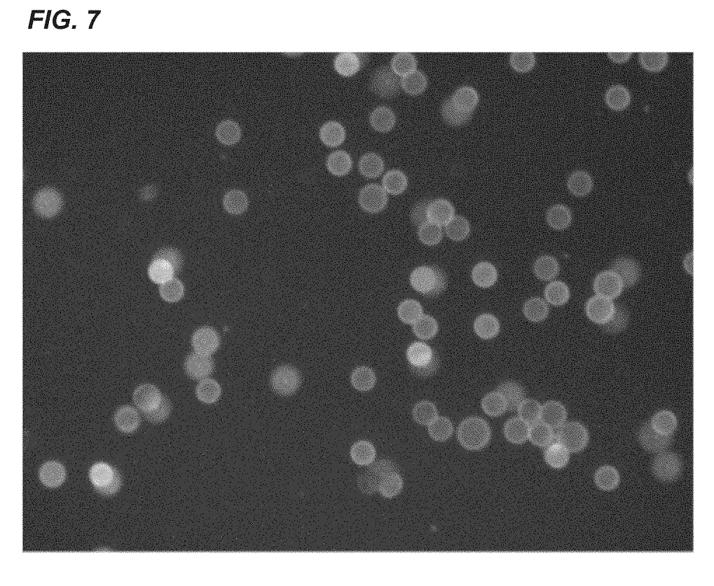

To demonstrate activity of the detection system, the prepared QD-SAV conjugate was used to detect biotin molecules on the surface of polystyrene spheres. In an Eppendorf tube, 10 μl of biotin spheres (Spherotech 1%, ˜7 microns) was mixed with 10 μl of the conjugate prepared as described above and incubated for 30 min at room temperature, then washed with 1 ml PBS and pelleted using a microcentrifuge (2000 rpm/5 min). The final spheres were observed using a fluorescent microscope (Olympus BX51) with a violet single bandpass excitation filter. As shown in FIG. 7, the SAV-QD conjugate is specifically bound to the surface of the biotin spheres. In this case both red and green emitting in vivo compatible water dispersible cadmium-free QD nanoparticles were conjugated to streptavidin and used to stain biotinylated polymer spheres. The plain non-biotinylated spheres when treated in the same manner did not show any staining, indicating that the binding of the QD is specific and attributed to the SAV functionality on the QD nanoparticle.

Covalent Conjugation of In Vivo Compatible Water Dispersible Cadmium Free QDs with Trastuzumab:

In Eppendorf tubes, 1.2 mg carboxyl-functionalised, water-soluble QDs were mixed with 100 μl MES activation buffer (i.e. 25 μl of 50 mg/ml stock into 100 μl MES). The MES buffer is prepared as a 25 mM solution (2-(N-morpholino) ethanesulfonic acid hemisodium salt MES), Sigma Aldrich) in DI water, pH 4.5. To this was added 33 μl of a fresh EDC solution (30 mg/ml stock in DI water, 1-ethyl-3-(3-dimethylaminopropyl) carbodiimide hydrochloride (EDC), Fisher Scientific) and the solution was mixed. To this was added 4 μl of fresh sulfo-NHS (100 mg/ml stock, ThermoFisher Scientific, in DI water) and the solution was mixed. NanoSep 300K filters (PALL NanoSep 300K Omega ultrafilters) were pre-wetted in 100 μl MES. The MES/EDC/Sulfo-NHS/QD solution was added to the NanoSep 300K filter and topped up 500 μl with MES. The filter was centrifuged at 5000 rpm/15 min. The retained dots were re-dispersed in 50 μl activation buffer and transferred to an Eppendorf tube containing 10 μl of trastuzumab (HERCEPTIN®, 100 mg/ml stock in a 25 mM solution of HEPES buffer, pH 8.5)+40 μl HEPES, pH 8.5. The solution was mixed well and incubated at RT overnight (around 16-18 hours). The solution was quenched with 16 μl of 6-amino caproic acid (6AC) (19.7 mg/100 mM). Note that quenching could be alternatively conducted with other compounds having a primary amine, but 6AC was selected for this embodiment because it has a COOH and can maintain the colloidal stability of the product. The solution was transferred to a pre-whetted Nanosep 300K filter (100 μl 1×PBS) and topped-up to the 500 μl line with 1×PBS. Excess SAV was removed by three cycles of ultrafiltration using Nanosep 300K filters and 1× PBS buffer. Each cycle of centrifugation was 5000 rpm for 20 min with re-dispersal with ˜400 μl of 1×PBS after each cycle. The final concentrated was re-dispersed in 100 μl PBS. FIGS. 6A and B demonstrate red nanoparticles conjugated to the anti-HER2 mAb (trastuzumab, Herceptin®) in binding to and identifying cells expressing HER2. In FIG. 6A the red nanoparticles conjugated to anti-HER2 mAb were used to treat HER2 positive 4T1 cells. FIG. 6B shows the same treatment with control HER2 negative 4T1 cells showing little fluorescence.

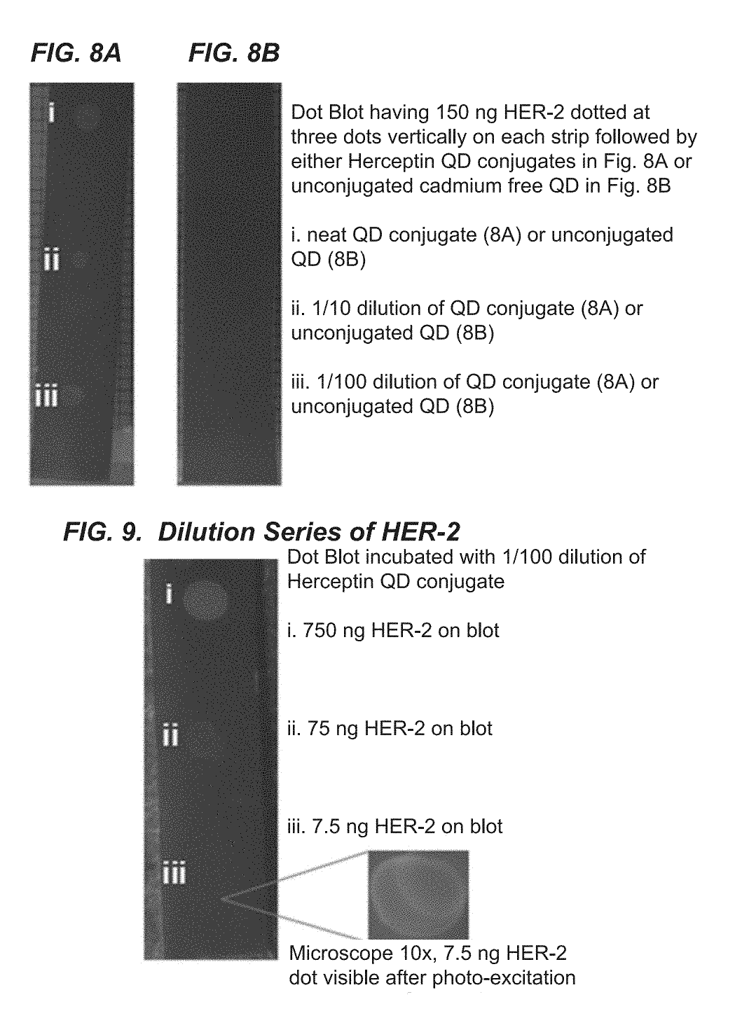

Experiments were undertaken to insure that the in vivo compatible water dispersible cadmium-free QDs do not bind non-specifically. For the experiments having results shown in FIGS. 8A and 8B, dot blots were performed. Aliquots (1 μl) of purified HER2 at a concentration of 0.15 mg/ml were first dotted vertically on three spots on each of two strips. FIG. 8A shows the results of adding differing concentrations of HERCEPTIN® QD conjugates to the strips previously dotted with purified HER2: i. neat HERCEPTIN® QD conjugate, ii. 1/10 dilution of HERCEPTIN® QD conjugate, iii. 1/100 dilution of HERCEPTIN® QD conjugate. FIG. 8B show the results of adding differing concentrations of the unfunctionalized in vivo compatible water dispersible cadmium-free QDs to the strips previously dotted with purified HER2. The results of the dot blot experiments showed that QD conjugated to Herceptin bound to HER2 and were readily detectable at relatively high dilutions when induced to fluoresce by exposure of the strips to excitatory light. Strips lacking prior dotting with HER2 did not cause binding by unconjugated QDs. FIG. 9 shows the results of a dilution series of HER2 dotted on a dot blot strip. The results showed that as little as 7.5 ng of HER2 dotted on a dot blot strip could be readily detected after incubation with a HERCEPTIN® QD conjugate.

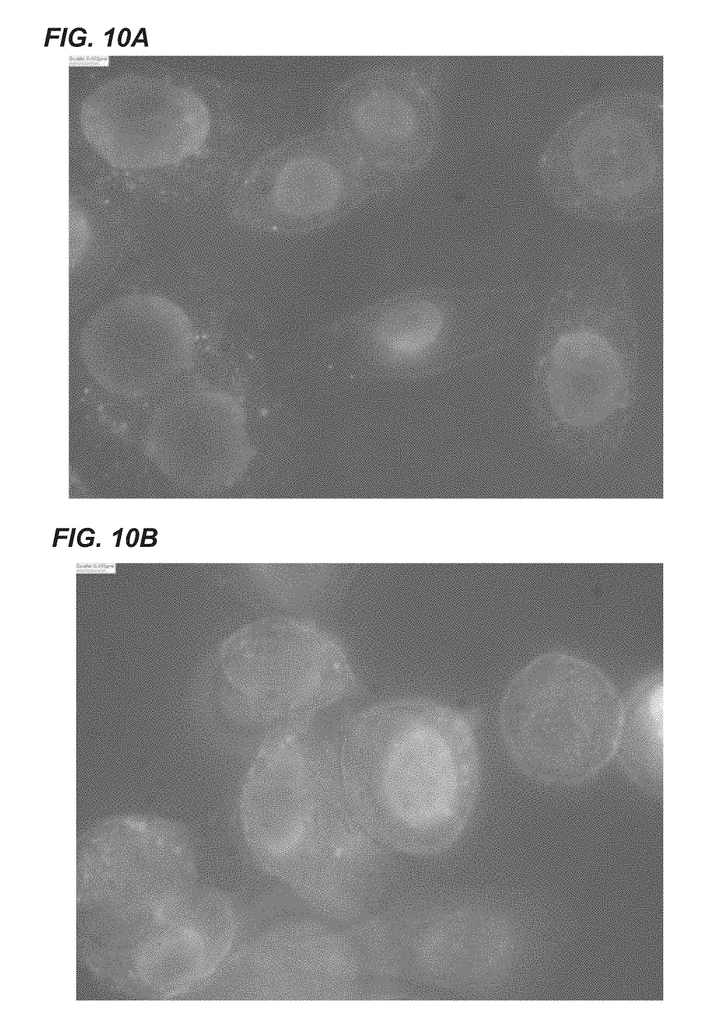

FIGS. 10A and 10B show the results of binding in vivo compatible water dispersible cadmium-free QDs to a SK-BR-3 cell line with and without a targeting moiety. The SK-BR-3 line is a human breast cancer cell line that overexpresses HER2. SK-BR-3 cells were cultured in McCoy's Modified Media with 10% FCS and treated overnight with 50 μg/mL of passive or Trastuzumab functionalized cadmium-free QDs that fluoresce at 630 nm. The excitation source used to visualize the cells was a mercury arc lamp (Osram HBO50W/AC L1 Short Arc, 2000 Lumens) and a DAPI light filter cube (excitation band is 380-450 nm) with broad emission bandpass. Cells after staining were visualised on an Olympus BX51 fluorescence microscope. Nuclei were counter stained with Hoechst 33342. FIG. 10A shows the results of treating SK-BR-3 cells with unfunctionalized in vivo compatible water dispersible cadmium-free QDs. FIG. 10B shows the results of treating SK-BR-3 cells with trastuzumab functionalized in vivo compatible water dispersible cadmium-free QDs and shows greatly enhanced uptake of QDs when functionalized with anti-HER2.

### Example 4

### MULTI-Ligand Theranostic Nano-Devices Directed Against Pancreatic Cancer

The rationale for choosing multiple ligands is to enable the targeting of a broad spectrum of the receptors associated with tumor cells. In certain embodiments, the specificity of the multiple ligands include at least one ligand specific for binding a target selected from the group consisting of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

In one particular embodiment the tumor is pancreatic cancer. About 70% of pancreatic tumors express EGFR as previously discussed, and this is overlapped with about 80% that express PD-L1.

Activated cytotoxic T cells, B cells and myeloid cells express a cell surface receptor called Programmed Death-1 (PD-1) receptor. PD-1 is an immune checkpoint receptor that modulates activation or inhibition of cell activity by these cells. The PD-1 receptor has two ligands, programmed death ligand-1 (PD-L1) and programmed death ligand-2 (PD-L2). PD-L1 also known as cluster of differentiation 274 (CD274) or B7 homolog 1 (B7-H1). Engagement of PD-L1 and PD-L2 on target cells with PD-1 receptors on activated T cells delivers an inactivating signal to the T-cell including by inhibiting TCR-mediated activation of IL-2 production and T cell proliferation. Normally the interaction between PD-1 on cytotoxic T cells and PD-L1 and PD-L2 on target cells may be useful to suppress the immune system during pregnancy and to prevent autoimmune disease. However, tumor cells exploit this pathway by over-expressing PD-L1 thus inhibiting the T-cell mediated anti-tumor response. Tumor infiltrating immune cells also express PD-L1 potentially resulting in inhibition of activated T-cells in the tumor microenvironment.

Of those pancreatic tumors expressing PD-L1, 20% have high upregulation of PD-L1 expression and these tumors tend to be highly invasive and recurrent. Thus, in one embodiment a bispecific nano-device is provided for the detection and treatment of cancers that express EGFR and PD-L1 based on QDs. The nano-device combines water soluble fluorescent QDs with two immunotherapeutic monoclonal antibodies (mAbs), an anti EGFR mAb and an anti-PD-L1 mAb.

In one embodiment the anti-EGFR mAb is cetuximab and the anti-PD-L1 mAb is atezolizumab. FIG. 4A and FIG. 4B present schematics for multi-ligand nano-devices and mechanisms of action on cancer cells. In the generation of such multi-ligand devices, desired amounts of selected antibodies such as for example cetuximab and atezolizumab are mixed and then reacted with carboxyl functionalized QDs using standard EDC chemistry. Other chemistries like Suzuki-Miyaura cross-coupling (SMCC), or aldehyde based reactions can be used as well.

The utilization of both cetuximab and atezolizumab will ensure the targeting universality of the nano-device against all pancreatic cancerous lesions and overcome the heterogeneity within the cancerous spread. In one embodiment the multi-ligand nano-device is utilized in preoperative diagnosis with minimally invasive endoscopy. In other embodiments, the multi-ligand device is utilized as an intra-operative and post-operative detection and follow up agent. The use of non-toxic water soluble QDs in this approach is useful not only due to their brightness and photo-stability but also to enable the attachment of at least two different targeting molecules, a feature that cannot be achieved using normal labelling dyes.

In certain embodiments, a multi-ligand nano-device is provided that supports minimally invasive molecular imaging and detection of low- and high-risk pancreatic cancers, leading to early diagnosis and treatment while reducing unnecessary surgical resections.

In such embodiments, the multi-ligand nano-device is injected into the circulation or into the abdomen and allowed to concentrate in the presumed tumor. A laparoscopic device is introduced into the abdomen that includes a light source for inducing fluorescence of the QDs and an imaging camera. Clinical detection and appropriate management of precursor lesions is an important strategy to reduce mortality from pancreatic cancer. An added benefit of the nano-devices disclosed herein is that, unlike with small dyes, photodynamic therapy can be applied. Excitation of the QDs in vivo results in induced apoptosis of cells bound by the functionalized QDs.

The device and methods may be used for other types of malignancies to increase the arsenal of available options for cancer treatment. Each QD may be equipped with more than one specific binding tag, forming multi-ligand nanodevices including but not limited to bi- or tri-specific nano-devices with maximum binding probability, and thus highest detection efficiency. The same concept of the described nano-device directed to pancreatic cancers and other cancers where EGFR and PD-L1 are over-expressed can be used to target other types of cancers by using the relevant mAbs or their derivatives.

In other embodiments, at least one of the target specific ligands is specific for a target selected from the group consisting of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

Human Epidermal Growth factor receptor 2 (HER2) is also known as CD340, proto-oncogene Neu, or ERBB2 and is a member of the epidermal growth factor receptor family. The ERBB2 gene is over expressed in a number of cancers including breast, uterine, gastric, stomach and salivary gland cancers. The monoclonal antibody monoclonal antibody trastuzumab (HERCEPTIN®) is currently used in treatment of HER2 positive breast cancer.

Carcinoembryonic Antigens (CEA) are glycosyl phosphatidyl inositol (GPI) cell-surface-anchored glycoproteins characterized immunologically as members of CD66a-f. CEA molecules are involved in cell adhesion, are normally not produced after birth and are thus present in very low levels in healthy adults. CEA levels are particularly elevated in colorectal cancer but may also be elevated in gastric, pancreatic, lung, breast and medullary thyroid carcinoma.

Carbohydrate antigen 19-9 (CA19-9), aka cancer antigen 19-9 or sialylated Lewis (a) antigen, is a Sialyl-Lewis^{A} carbohydrate antigen detected with the CA19-9 antibody that is involved in cell-to-cell recognition and is associated with advanced gastrointestinal cancers including colorectal and pancreatic cancers.

Cancer Antigen-125 (CA125) is also known as mucin 16 (MUC16) participates in cell-to-cell interactions that enable metastasis and is a biomarker for ovarian cancer among others.

CD20, Cluster of Differentiation 20, is an activated-glycosylated phosphoprotein encoded by the MS4A1 gene and expressed on the surface of B-cells but not early pro-B cells or plasma cells. CD20 is expressed in B-cell lymphoma, hairy cell leukemia, B-cell chronic lymphocytic leukemia and by melanoma stem cells among others. A number of monoclonal antibodies used in the treatment of B cell lymphomas and leukemias are directed to CD20 including rituximab, obinutuzumab, ibritumomab, and tositumomab.

CD25, Cluster of Differentiation 25, is the interleukin-2 receptor alpha chain (IL2RA) protein present on activated T and B cells and certain other cells and is expressed in most B-cell neoplasms, some acute nonlymphocytic leukemias, neuroblastomas, and mastocytosis and by tumor infiltrating lymphocytes. The humanized monoclonal antibody, daclizumab, is directed to CD25 but is currently FDA approved for multiple sclerosis only.

CD30, Cluster of Differentiation 30, a.k.a. TNFRSF8, is a tumor necrosis factor receptor family protein expressed by activated T and B cells. CD30 is expressed in anaplastic large cell lymphoma and embryonal carcinoma. The humanized monoclonal antibody, brentuximab, is FDA approved for treatment of Hodgkins lymphoma and systemic anaplastic large cell lymphoma.

CD33, Cluster of Differentiation 33, a.k.a. sialic acid binding Ig-like lectin 3 (SIGLEC-3) is an adhesion molecule of myelomonocytic-derived cells that mediates sialic-acid dependent binding to cells and preferentially binds to alpha-2,6-linked sialic acid. The humanized monoclonal antibody gemtuzumab binds to CD33. The combination of gemtuzumab and the calicheamicin cytotoxic agent ozogamicin is used to treat acute lymphoplastic leukemia.

CD52, Cluster of Differentiation 52, a.k.a. Cambridge Pathology Antigen-1 (Campath-1 antigen) is a small cell surface glycoprotein. CD52 is associated with certain types of lymphoma and is targeted by the monoclonal antibody alemtuzumab.

CD73, Cluster of Differentiation 73, a.k.a. ecto-5′-nucleotidase (NT5E), is a 70 kDa cell surface enzyme found in most tissues but appears to be upregulated in tumor cells, including in colon, lung, pancreas and ovarian carcinomas where it acts to impair antitumor T cell responses.

CD109, Cluster of Differentiation 109, is a glycosyl phosphatidylinositol (GPI)—linked glycoprotein that is involved in transforming growth factor beta binding. CD109 is typically found on the cell surface of platelets, activated T-cells, and endothelial cells but is expressed by CD34+ acute myeloid leukemia cells and is over expressed by pancreatic ductal carcinoma cells.

Vascular Endothelial Growth Factor A (VEGFA) is a dominant inducer of blood growth and is expressed in adults during organ remodeling and wound healing. However, VEGF is also pathogenic in tumor angiogenesis, diabetic retinopathy, and age-related macular degeneration. The humanized monoclonal antibody bevacizumab (AVASTIN®) is used to treat for colon cancer, lung cancer, glioblastoma, and renal-cell carcinoma as well as age-related macular degeneration.

Cytotoxic T-lymphocyte-associated protein 4 (CTLA-4), aka CD152 is an immune checkpoint protein receptor that down regulates immune responses. The monoclonal antibody ipilimumab activates the immune system by binding to CTLA-4 and is FDA approved in the retreatment of melanoma with clinical trials ongoing in the treatment of other cancers.

Receptor Activator of Nuclear Factor Kappa-B (RANK) Ligand (or RANKL) is also known as tumor necrosis factor ligand superfamily member 11 (TNFSF11), TNF-related activation-induced cytokine (TRANCE), osteoprotegerin ligand (OPGL), and osteoclast differentiation factor (ODF).

The advantages of the multi-specific nano-device including but not limited to bispecific nano-device including EGFR and PD-L1 specificities can be summarized as follows:
Maximum tumor labeling due to the fluorescence properties of QDs and the high tagging probability due to the functionalization with two or more different binding tags,
EGFR inhibition due to cetuximab binding to EGFR and inhibition of PD-L1 mediated down regulation of activated T cells,
Synergized tumor uptake due to EPR (Enhanced Permeability and Retention) effect contributed by the nano size of the conjugate,
Tumor phototherapeutic effect due to the ability of the QDs to generate singlet oxygen or condense energy in the form of heat that can trigger cell suicidal cascade (apoptosis).

The combination of the above four mechanisms are believed to provide synergistic effects, which could result in a very efficient treatment for EGFR and PD-L1 expressing cancers and lower side effects from the ability to deliver lower doses of QD bound cetuximab and atezolizumab.

### STATEMENTS OF THE DISCLOSURE

Statement 1. A nanoparticle conjugate (nano-device) having multiple target specificities comprising surface modified, water soluble quantum dot (QD) nanoparticles each of which are chemically conjugated to at least two different target specific ligands.

Statement 2. The nano-device of Statement 1, wherein at least one of the target specific ligands is specific for a target selected from the group consisting of EGFR, PD-L1, PD-L2, HER2, CEA, CA19-9, CA125, telomerase proteins and subunits, CD20, CD25, CD30, CD33, CD52, CD 73, CD109, VEGF-A, CTLA-4, and RANK ligand.

Statement 3. The nano-device of Statement 2, wherein at least one target specific ligand is a monoclonal antibody direct to EGFR.

Statement 4. The nano-device of Statement 2, wherein at least one target specific ligand is monoclonal antibody direct to PD-L1.

Statement 5. The nano-device of Statement 2, wherein the surface modified, water soluble QD nanoparticle is chemically conjugated to at least two ligands, one of which is specific for binding to EGRF and one of which is specific for binding to PD-L1.

Statement 6. The nano-device of Statement 5, wherein the surface modified, water soluble QD nanoparticle is chemically conjugated to at least two ligands including cetuximab and atezolizumab.

Statement 7. A mixture of nanoparticle conjugates (nano-devices) comprising at least two populations of nano-devices including a first target specific population comprising surface modified, water soluble quantum dot (QD) nanoparticles derivatized with a first target specific antibody and a second target specific population comprising surface modified, water soluble quantum dot (QD) nanoparticles derivatized with a second target specific antibody.

Statement 8. The mixture of Statement 7, wherein the first target specific antibody is specific for EGFR and the second target specific antibody is specific for PD-L1.

Statement 9. The nano-device of any one of Statement 1 through Statement 6, wherein the surface modified non-toxic, water soluble QD nanoparticle is cadmium-free and comprises semiconductor materials selected from the group of materials consisting of ZnS, ZnSe, ZnTe, InP, InAs, InSb, AlP, AlS, AlAs, AlSb, GaN, GaP, GaAs, GaSb, PbS, PbSe, AgInS₂, AgInS₂/ZnS, Si, Ge and alloys and doped derivatives thereof.

Statement 10. The nanodevice of Statement 9, wherein the cadmium-free surface modified non-toxic, water soluble QD nanoparticle comprises a core formed of one of the materials and one or more shells of another of the materials.

Statement 11. The nanodevice of Statement 10, wherein the cadmium-free surface modified non-toxic, water soluble QD nanoparticle is a core/multi-shell QD.

Statement 12. The nano-device of any one of Statement 1 through Statement 6, wherein the surface modified, water soluble quantum dot (QD) nanoparticle comprises a ligand interactive agent and a surface modifying ligand.

Statement 13. The nano-device of Statement 12, wherein the surface modified QD nanoparticle is formed by chemical addition of the ligand interactive agent and the surface modifying ligand to the QD in a solution comprising hexamethoxymethylmelamine. In certain embodiments the solution is a non-polar solution. In particular embodiments the non-polar solution is toluene.

Statement 14. The nano-device of Statement 12, wherein the ligand interactive agent is a C₈₋₂₀ fatty acid and esters thereof.

Statement 15. The nano-device of Statement 12, wherein the surface modifying ligand is a monomethoxy polyethylene oxide.

Statement 16. A method of simultaneously inducing cell death, tumor shrinkage, and fluorescent labelling of tumor cells by administering a nanoparticle conjugate comprising a non-toxic water soluble quantum dot (QD) nanoparticle s chemically conjugated to a monoclonal antibody that is directed to cell surface moiety over-expressed by tumor cells.

Statement 17. The method of Statement 16, wherein the cell surface moiety is an epidermal growth factor receptor (EGFR).

Statement 18. The method of Statement 16, wherein the tumor is a pancreatic tumor.

Statement 19. A nanoparticle conjugate (nano-device) composed of two or more different therapeutic antibodies attached to a quantum dot (QD) nanoparticle, wherein the different therapeutic antibodies have different target specificities.

Statement 20. The nano-device of Statement 19, wherein the QD nanoparticle is chemically conjugated an antibody directed against EGFR and to an antibody directed against PD-L1.

Statement 21. A method of detecting or therapeutically modulating biological moieties comprising:

allowing a composition comprising target specific derivatized cadmium-free water soluble quantum dots (QDs) to interact physically with a biological target, wherein the cadmium-free water soluble quantum dots have been surface modified using the melamine cross-linker hexamethoxymethylmelamine (HMMM) to include one or more functional groups selected from the group consisting of COOH, NH₂, SH, OH, sulfonate, phosphate, azide, allyl, silyl and PEG chains and are further derivatized via the one or more functional groups with a plurality of different target specific ligands;

stimulating emission from the target specific derivatized cadmium-free water soluble QDs via application of light at an excitatory wavelength for the QD; and

recording and/or imaging a spectral emission of interactions between the composition and the biological target.

Statement 22. The method of Statement 21, wherein the ligands are selected from one or more of the group consisting of antibodies, streptavidin, nucleic acids, lipids, saccharides, drug molecules, proteins, peptides, and amino acids.

Statement 23. The method of Statement 21, wherein the detecting is used for imaging and detecting one or more of angiogenesis, tumor demarcation, tumor metastasis, diagnostics in vivo, and lymph node progression.

Statement 24. The method of Statement 21, wherein the detecting is used in one or more of immunochemistry, immunofluorescence, DNA sequence analysis, fluorescence resonance energy transfer, flow cytometry, fluorescence activated cell sorting, and high-throughput screening.

Statement 25. The method of any one of Statement 21 through Statement 24, wherein the plurality of different target specific ligands are ligands having specificity for targets selected from the group consisting of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

Statement 26. A multi-ligand nano-device comprising cadmium-free water soluble quantum dots derivatized with a plurality of ligand populations, each population having a different target specificity.

Statement 27. The multi-ligand nano-device of Statement 26, comprising at least one target specific antibody ligand and a further ligand selected from streptavidin, nucleic acids, lipids, saccharides, drug molecules, proteins, peptides, and amino acids where the antibody ligand acts as a targeting ligand to deliver the further ligand.

Statement 28. The multi-ligand nano-device of Statement 26, comprising at least two target specific antibody ligands at least one of which is a ligand having specificity for targets selected from the group consisting of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

Statement 29. A multi-ligand nano-device manufactured for use as a medicament for detecting and treating cancer, wherein the multi-ligand nano-device comprises a cadmium-free water soluble quantum dot (QD) derivatized with a plurality of ligand populations, each ligand population having specificity for a different target overexpressed on cancer cells.

Statement 30. The multi-ligand nano-device of Statement 29, wherein the device is utilized in preoperative or intraoperative diagnosis with minimally invasive endoscopy or laparoscopy.

Statement 31. The multi-ligand nano-device of Statement 29 or Statement 30, wherein the multi-ligand nano-device is adapted for injection into the circulation or into the abdomen and concentration in tumor cells relative to normal cells.

Statement 32. The multi-ligand nano-device of Statement 31, wherein the multi-ligand nano-device is adapted for injection into the circulation or into the abdomen and concentration in tumor cells relative to normal cells by surface modification with monomethoxy polyethylene oxide.

Statement 33. The multi-ligand nano-device of Statement 29 or Statement 30, wherein the concentrated multi-ligand nano-device can be induced to fluoresce by exposure to a QD excitatory light source applied endoscopically or laparoscopically, said fluorescence detectable by an imaging camera.

Statement 34. The multi-ligand nano-device of Statement 29 or Statement 30, wherein the different targets overexpressed on cancer cells are selected from EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

Statement 35. The multi-ligand nano-device of Statement 34 wherein the different targets overexpressed on cancer cells are EGFR and PD-L1.

Statement 36. The multi-ligand nano-device of Statement 29 or Statement 30, wherein the cadmium-free water soluble quantum dots have a quantum yield of at least 50%.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. A nanoparticle conjugate (nano-device) having multiple target specificities comprising surface modified, water soluble quantum dot (QD) nanoparticles each of which are chemically conjugated to at least two different target specific ligands.

2. The nano-device of claim 1, wherein at least one of the target specific ligands is specific for a target selected from the group consisting of EGFR, PD-L1, PD-L2, HER2, CEA, CA19-9, CA125, telomerase proteins and subunits, CD20, CD25, CD30, CD33, CD52, CD 73, CD109, VEGF-A, CTLA-4, and RANK ligand.

3. The nano-device of claim 2, wherein at least one target specific ligand is a monoclonal antibody direct to EGFR.

4. The nano-device of claim 2, wherein at least one target specific ligand is monoclonal antibody direct to PD-L1.

5. The nano-device of claim 2, wherein the surface modified, water soluble QD nanoparticle is chemically conjugated to at least two ligands, one of which is specific for binding to EGRF and one of which is specific for binding to PD-L1.

6. The nano-device of claim 1, wherein the surface modified non-toxic, water soluble QD nanoparticle is cadmium-free and comprises semiconductor materials selected from the group of materials consisting of ZnS, ZnSe, ZnTe, InP, InAs, InSb, AlP, AlS, AlAs, AlSb, GaN, GaP, GaAs, GaSb, PbS, PbSe, AgInS₂, AgInS₂/ZnS, Si, Ge and alloys and doped derivatives thereof.

7. The nano-device of claim 1, wherein the surface modified, water soluble quantum dot (QD) nanoparticle comprises a ligand interactive agent and a surface modifying ligand.

8. The nano-device of claim 7, wherein the surface modified QD nanoparticle is formed by chemical addition of the ligand interactive agent and the surface modifying ligand to the QD in a solution comprising hexamethoxymethylmelamine.

9. The nano-device of claim 7, wherein the ligand interactive agent is a C₈₋₂₀ fatty acid and esters thereof.

10. The nano-device of claim 7, wherein the surface modifying ligand is a monomethoxy polyethylene oxide.

11. A method of detecting and/or therapeutically modulating biological moieties comprising:
allowing a composition comprising target specific derivatized cadmium-free water soluble quantum dots (QDs) to interact physically with a biological target, wherein the cadmium-free water soluble quantum dots have been surface modified using the melamine cross-linker hexamethoxymethylmelamine (HMMM) to include one or more functional groups selected from the group consisting of COOH, NH₂, SH, OH, sulfonate, phosphate, azide, allyl, silyl and PEG chains and are further derivatized via the one or more functional groups with a plurality of different target specific ligands;
stimulating emission from the target specific derivatized cadmium-free water soluble QDs via application of light at an excitatory wavelength for the QD; and
recording and/or imaging a spectral emission of interactions between the composition and the biological target.

12. The method of claim 11, wherein the plurality of different target specific ligands are ligands having specificity for targets selected from the group consisting of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

13. A multi-ligand nano-device comprising cadmium-free water soluble quantum dots derivatized with a plurality of ligand populations, each population having a different target specificity.

14. The multi-ligand nano-device of claim 13, comprising at least one target specific antibody ligand and a further ligand selected from streptavidin, nucleic acids, lipids, saccharides, drug molecules, proteins, peptides, and amino acids where the antibody ligand acts as a targeting ligand to deliver the further ligand.

15. The multi-ligand nano-device of claim 13, comprising at least two target specific antibody ligands at least one of which is a ligand having specificity for targets selected from the group consisting of EGFR, PD-L1, PD-L2, HER2, CEA, CA19-9, CA125, telomerase proteins and subunits, CD20, CD25, CD30, CD33, CD52, CD73, CD109, VEGF-A, CTLA-4, and RANK ligand.

16. The multi-ligand nano-device of claim 13, wherein the multi-ligand nano-device is adapted for injection into the circulation or into the abdomen and for concentration in tumor cells relative to normal cells by surface modification with monomethoxy polyethylene oxide.

17. The multi-ligand nano-device of claim 16, wherein the targets comprise EGFR and PD-L1.

18. The multi-ligand nano-device of claim 13, wherein the cadmium-free water soluble quantum dots have a quantum yield of at least 50%.

19. A mixture of nanoparticle conjugates (nano-devices) comprising at least two populations of nano-devices including a first target specific population comprising surface modified, water soluble quantum dot (QD) nanoparticles derivatized with a first target specific antibody and a second target specific population comprising surface modified, water soluble quantum dot (QD) nanoparticles derivatized with a second target specific antibody.

20. The mixture of claim 19, wherein the first target specific antibody is specific for EGFR and the second target specific antibody is specific for PD-L1.
